(19) United States
(12) Patent Application Publication
(10) Pub. No.: US 2016/0151591 A1
(43) Pub. Date: Jun. 2, 2016

(54) Anesthesia Station for Laboratory Animals and Method for Determining the Organohalogen Saturation Rate of the Filters of Such an Anesthesia Station

(71) Applicant: ANESTEO, Lunel (FR)

(72) Inventors: Thomas Le Bars, Villetelle (FR); Sebastien Caron, Bussy Les Daours (FR)

(73) Assignee: Anesteo, Lunel (FR)

(21) Appl. No.: 14/907,075

(22) PCT Filed: Jun. 5, 2014
(86) PCT No.: PCT/FR2014/051341

§ 371 Date: Jan. 22, 2016

(30) Foreign Application Priority Data
Jul. 23, 2013 (FR) 1357222

(51) Int. Cl. A61M 16/00; A61M 16/01; A61M 16/10; A61D 7/04

(52) U.S. Cl. CPC A61M 16/0051; A61D 7/04; A61M 16/0093; A61M 16/01; A61M 16/105; A61M 2205/7581; A61M 2230/437; A61M 2016/0033; A61M 2205/502; A61M 2205/3306; A61M 2205/60; A61M 2205/581; A61M 2205/583

(57) ABSTRACT

Provided herein is an anesthesia station for at least one laboratory animal, including an anesthesia area connected to at least one peripheral receiving the laboratory animal. The anesthesia area is able to supply the peripheral with, halogenated anesthetic gases. The station further includes a system for aspirating the halogenated gases from the peripheral and at least one filter having a determined organohalogen concentration retention capacity. The filter is arranged in such a way as to be crossed by the halogenated anesthetic gases from the peripheral during operation of the aspiration system. The station also includes a system for the automatic determination in real-time of the organohalogen saturation rate of the filter.

### TECHNICAL FIELD OF THE INVENTION

The invention relates to the field of the use of gaseous anesthetics intended for laboratory animals (small animals), such as halogenated anesthetic gases.

The invention relates more specifically to an anesthesia station for laboratory animals with filter capture of the halogens contained in the anesthetic gases, and a method for determining the organohalogen saturation rate of the filter provided for said anesthesia station.

### PRIOR ART

Conventionally, an anesthesia station is responsible for distributing halogenated gases for the purpose of anesthetizing laboratory animals, as well as for aspirating and filtering gases distributed in order to protect users of the station.

The actual operation of anesthesia conventionally comprises two main phases: a first phase, called the induction phase, for putting the animal to sleep or causing it to lose consciousness; and a second phase, called the maintenance phase, for keeping the animal asleep. The induction phase comprises delivering a large volume of highly concentrated anesthetic agents to the interior of a box, called an induction box or chamber, in order to rapidly put the animal to sleep. The maintenance phase, in turn, comprises delivering the gaseous mixture at a lower rate and lower concentration to the anesthetized animals, so as to keep them asleep. The gas in this case is delivered with the use of masks.

Then, in the anesthesia stations of the prior art, the gas is aspirated and filtered by filters that retain organohalogens, the filters being placed in the aspiration circuit at the outlet from the induction chamber. U.S. Pat. No. 6,776,158, cited by way of example, describes an anesthesia system for laboratory animals that comprises an activated carbon filter.

Furthermore, anesthesia stations are customarily equipped with a suction plate in order to be able to locally aspirate the halogenated gases, especially in the masks.

The anesthesia stations of the prior art, however, have a drawback in that the filters used must be regularly checked in order to verify that the filters are not saturated or are not approaching the saturation point with organohalogens. Indeed, a filter that is completely saturated with organohalogens no longer performs its function, and consequently exposes the users to the anesthetizing gas when the anesthesia station is operated.

In order to alleviate this drawback in some measure, some anesthesia stations of the prior art provide visual and/or sound-based alert systems. The anesthesia system of the aforementioned patent, for example, is provided with a device for determining the rate of absorption of organohalogens in the filter by measuring the rate of organohalogens at the filter exit, said device being connected to means for an alarm in case a predetermined absorption rate threshold is exceeded. Such an anesthesia system, however, presents a drawback in that it does not enable users to check the state of the filter (whether the filter is only slightly saturated or is close to the point of saturation) at any moment during the course of the anesthesia operation, and the users are thus only notified at a predetermined threshold level. The users also do not have the ability to see the state of the filter before the anesthesia operation is started. The user is thus unable to assess the state of the filter before initiating the anesthesia or during the anesthesia operation, and therefore cannot be certain of not being exposed to the organohalogens during the course of the anesthesia if the filter is saturated during that period.

The invention is designed to remedy these problems by suggesting an anesthesia station and method therefor which make it possible to automatically check the rate of saturation of the filter before the start of the anesthesia and during the anesthesia, and in a manner that is reliable and secured for the user.

### OBJECT OF THE INVENTION

To that end, and according to a first aspect, the invention suggests an anesthesia station for one or more laboratory animals, which comprises: an anesthesia area connected to at least one peripheral receiving the laboratory animal, said anesthesia area being able to deliver halogenated anesthetic gases to said peripheral; means for aspirating halogenated gases from the peripheral; at least one filter having a predetermined organohalogen concentration retention capacity, said filter being arranged in such a way as to be crossed by the halogenated anesthetic gases originating from the peripheral during operation of the aspirating means; and a system for automatically determining, in real time, the organohalogen saturation rate of the filter, said system comprising: means for measuring instantaneous flow rates of the halogenated anesthetic gases passing through the filter at each given time interval; means for determining the concentration of the halogenated gases retained in the filter on the basis of the instantaneous flow rates of the halogenated anesthetic gases measured at each given time interval; and means for calculating the saturation rate of the filter on the basis of the concentrations of the halogenated anesthetic gases determined at each time interval and on the basis of the predetermined retention capacity of the filter.

Advantageously, the calculating means are configured to calculate the remaining organohalogen concentration capacity of the filter at a given moment.

Advantageously, the calculating means are configured to calculate an estimated time remaining for using the filter. This remaining time is determined on the basis of the identified usage protocol. More particularly, the remaining time is estimated on the basis of a history of usage data that is recorded in a memory of a control unit of the anesthesia station.

Advantageously, the anesthesia station comprises means for displaying data relating to the saturation rate of the filter, the remaining organohalogen concentration retention capacity of the filter, and/or the estimated remaining usage time of the filter.

Advantageously, the anesthesia area is connected to at least one peripheral device for distributing halogenated anesthetic gases.

Advantageously, the aspirating means and the filter are integrated into the anesthesia area. It may also be provided that the system for automatically determining the organohalogen saturation rate of the filter in real time is integrated into the anesthesia area.

Advantageously, the anesthesia station comprises a first type of peripheral, called an “induction” peripheral, which has the form of a hermetically sealed chamber. The anesthesia station may also comprise a second type of peripheral, called a “maintenance” peripheral, which has the form of a mask designed to receive a previously anesthetized laboratory animal. It may be provided that the anesthesia station comprises one or more additional peripheral(s) connected to the ventilating means. This peripheral may for example have the form of a suction plate for locally aspirating the halogenated gases at the level of a peripheral of the first type or of the second type. It may also be an optical imager, such as an imager that uses bioluminescence technology to identify and characterize the development of tumors in the animal.

Advantageously, the anesthesia station comprises a control unit 100 that controls the operation of the anesthesia area, said control unit controlling the stopping of the delivery of the halogenated anesthetic gases when the calculated saturation rate of the filter corresponds to the maximum saturation rate of the filter.

Advantageously, the control unit comprises calculating means for calculating the aspiration power delivered by the aspirating means based on the volume of anesthetic gas delivered to the peripheral.

Advantageously, the control unit comprises an RFID reader designed to communicate with an RFID tag carried by the filter.

Advantageously, the anesthesia area comprises visual and/or sound-based alert means coupled to the calculation unit.

The invention also relates to a method for determining the organohalogen saturation rate of a filter through which halogenated anesthetic gases flow, the filter having a predetermined organohalogen concentration retention capacity, said method being characterized in that it comprises the steps of:
measuring the flow of halogenated anesthetic gases at regular intervals of time;
determining the concentration of gas retained in the filter, by correlation with the flow measured at each time interval; and
calculating the saturation rate of the filter by summing the gas concentrations determined at each time interval and comparing them with the predetermined retention capacity of the filter.

Advantageously, the method comprises a step of determining the remaining organohalogen concentration retention capacity on the basis of data relating to the saturation rate of the filter as calculated at a time t, and relating to the organohalogen concentration retention capacity.

Advantageously, the method comprises a step of determining the estimated remaining usage time on the basis of data relating to the remaining organohalogen concentration retention capacity and relating to the mean flow rate of the halogenated anesthetic gases as calculated on the basis of the instantaneous flow rates of the halogenated gases as measured at each time interval.

Advantageously, the saturation rate of the filter is calculated on the basis of the sum of the concentration of gases determined at a given instant when the instantaneous flow is measured, with the incremented concentrations of the gases determined at each time interval.

### BRIEF DESCRIPTION OF THE DRAWINGS

Other objects and advantages of the invention will be made more apparent from the following description, with reference to the accompanying drawings, in which:

FIG. 1 is a block diagram of the anesthesia station according to the invention; and

FIG. 2 represents an overview of the control of the anesthesia station.

### DETAILED DESCRIPTION OF THE DRAWINGS

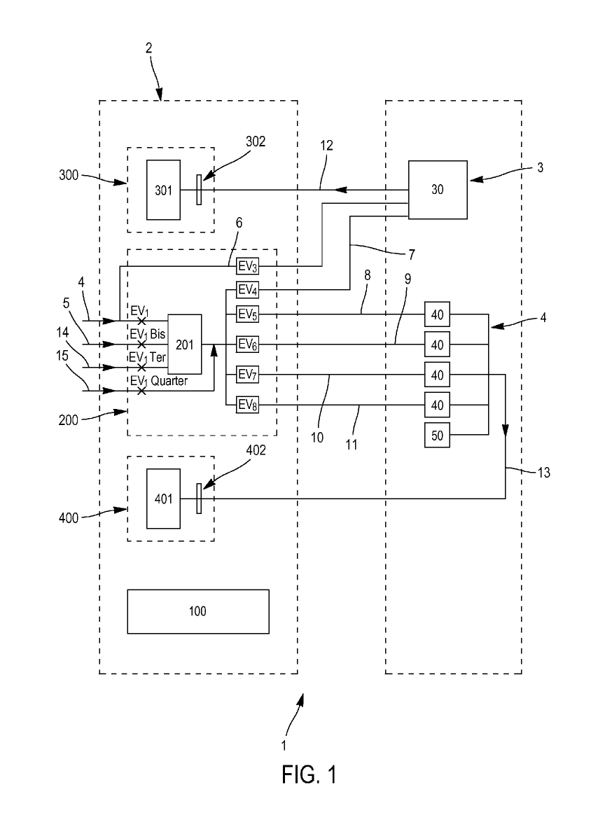

FIG. 1 represents an anesthesia station 1 for laboratory animals such as rodents.

The anesthesia station 1 comprises an anesthesia area 2 connected to peripherals 3, 4 that receive the animals. The anesthesia area 2 is responsible not only for distributing halogenated anesthetic gases in order to anesthetize the animals, but also for aspirating and filtering the gases distributed in the peripherals 3, 4.

In the illustrated embodiment, the peripherals 3, 4 comprise an induction chamber 30 for the phase of anesthesia called the induction phase, and masks 40 (there being four such masks in the example illustrated) for the phase of anesthesia called the maintenance phase. The induction chamber 30 has the form of a hermetically sealed container with a door or lid that is either fixed or movable between an open position—for allowing animals to be passed into the container—and a closed position. In addition to the induction chamber 30 and the masks 40, the anesthesia station 1 advantageously comprises a suction plate 50. Such a plate makes it possible to locally aspirate the halogenated gases at the level of a peripheral 3, 4, and in particular at the level of the masks 40.

In the example illustrated, the anesthesia station 1 comprises six peripherals, one of which is a suction plate. It shall be readily understood that the number of ports for the peripherals in the anesthesia area 2 may vary depending on the desired size of the station. In general, the induction chamber 30 constitutes the essential peripheral of the anesthesia station 1, and the other peripherals 40, 50 (masks 40 and suction plate 50) may vary from one station to another depending on the quantity, size, and type of animals to be anesthetized.

Though not shown, an anesthesia station comprising a plurality of induction chambers may also be provided.

The anesthesia area 2 comprises a distribution point 200 connected to each of the peripherals 3, 4 by a fluid injection circuit and two aspiration and filtration points 300, 400, one of which is connected to the peripheral of the induction phase (induction chamber 30) via a first fluidic aspiration circuit and the other of which is connected to the peripherals 40, 50 of the maintenance phase (masks 40 and suction plate 50) via a second fluidic aspiration circuit.

In the embodiment shown, the distribution point 200 of the anesthesia area 2 comprises, at the input, three fluidic distribution lines 4, 5, and 14, each line being designed to transport a gas toward an evaporator 201. Thus, the distribution point 200 comprises a first fluidic line 4 for distributing oxygen, a second fluidic line 5 for distributing air, and a third fluidic line 14 for distributing nitrous oxide. The gases that are distributed in this manner to the evaporator are carrier gases that will make it possible, either alone or mixed together, to evaporate an isoflurane- or sevoflurane-type anesthetic agent contained in the evaporator 201. The anesthetic agent, which is originally introduced in a liquid state into the evaporator 201 with a filling key, thus passes into the gaseous state under the action of the carrier gas(es) injected into the evaporator 201. In the illustrated embodiment, the fluidic line 4 is provided with a bypass 6 toward the induction chamber, in order to make it possible to deliver oxygen into the induction chamber 30 once the induction phase has been completed. The distribution lines are each provided with a solenoid valve EV1, EV1bis and EV1ter, and the bypass is provided with a solenoid valve EV3.

Advantageously, the distribution point 200 comprises a fourth fluidic line 15 for distributing carbon dioxide to the peripherals 3 or 4. The fluidic line 15 is connected to the outlet of the evaporator 201, and is provided with a solenoid valve EV1quater.

It will be readily understood that the distribution point is not limited to the above-described configuration, and the number of fluidic lines at the distribution point inlet may vary without departing from the scope of the invention. According to a minimal configuration, a distribution point that only distributes a single carrier gas—in this case, oxygen—may also be provided. A single fluidic line will then be needed, and the other lines will be optional.

The distribution point 200 comprises, at the outlet, five ports for connecting the anesthesia area 2 to the five peripherals 30, 40 that deliver the anesthetic gas to the animals (the induction chamber 30 and masks 40). The injection line 7 connecting the distribution point 200 to the induction chamber 30 is independent of the injection lines 8 to 11, which connect the distribution point 200 to the masks 40. As before, each of the distribution lines is provided with a solenoid valve EV4 to EV8.

As indicated above, the anesthesia station 1 comprises two aspiration/filtration points 300, 400, a first point 300 being connected to the induction chamber 30 and a second point 400 being connected to the masks 40 and to the suction plate 50. It will be readily understood that the number of aspiration/filtration points is not limited to two, and that an anesthesia station 1 may be provided that comprises one aspiration/filtration point shared by the two types of peripherals 30, 40, including the suction plate 50, or indeed more than two aspiration points. The latter configuration may particularly be provided for anesthesia stations that comprise a large number of peripherals.

Each of the aspiration/filtration points 300, 400 comprises a fan 301, 401 connected to the output of one of the peripherals 30, 40, 50 by an aspiration line. Thus, the induction chamber 30 is connected at the output to one of the fans 301 via a first aspiration line 12, whereas each of the masks and the suction plate 50 are joined to a second aspiration line 13 that connects them to the other fan 401. The first and second aspiration lines are independent of each other. The fans 301, 401 are controlled, and control the flow of gases entering into the peripherals.

In order to retain the halogenated fraction of the anesthetic gas, and thus to protect the users when the gas is being aspirated, the fluidic aspiration circuits connecting the induction chamber 30 and the maintenance peripherals 40 are each provided with an organohalogen retention filter 302, 402. More particularly, each of the filters 302, 402 is arranged in the aspiration lines 12, 13 between the peripheral in question (induction chamber 30, mask, or suction plate 50) and the associated fan 301, 401, so that the halogenated anesthetic gases pass through it when the associated fan is operated.

The number of filters is independent of the number of fans. Thus, one filter may be provided per peripheral. According to another configuration, one filter may be provided that is shared by the induction peripheral 3 and the maintenance peripherals 4.

Each of the filters 302, 402 is advantageously equipped with an RFID identification tag (not shown). Each of the filters 302, 402 thus has a unique identification number, which is associated (as will be described below) with an RFID interface 101, or a counter of a control unit 100 integrated in the anesthesia area 2. Thus, when the identification number of the filter is not known, a new counter is created and initialized by the control unit 100. If the number is known, then the associated counter is counted down periodically during the operation of the pump. The absence of a response from the tag signifies that the filter is absent. A message “Filter missing” is then displayed on a screen provided for this purpose, preferably on the anesthesia area 2. As will be discussed below, the absence of the filter or the completed countdown of the counter will prevent any operation of the anesthesia area 2 (i.e., any distribution of anesthetic gas).

Advantageously, the filter 302, 402 is a turbulent-flow honeycomb filter. The filter 302, 402 may be a molded carbon filter with a honeycomb structure in which the gases are absorbed by a reagent. The advantage of such a filter is optimized capture of the organohalogens due to the turbulent-flow technology.

To enable real-time control of the filter 302, 402 and ensure functionality thereof for the user of the anesthesia station 1, the anesthesia station 1 comprises a system for automatically determining the organohalogen saturation rate of the filter 302, 402 in real time. The system for automatically determining saturation rate is therefore designed to assess the wear rate of the filter 302, 402, and to warn the user where appropriate. To this end, the system for determining the saturation rate comprises: means for measuring instantaneous flow rates of the halogenated anesthetic gases passing through the filter 302, 402, at given time intervals; means for determining the concentration of the halogenated gases retained in the filter 302, 402 on the basis of the instantaneous flow rates of the halogenated anesthetic gases measured at each time interval; and means for calculating the saturation rate of the filter 302, 402 on the basis of the concentrations of the halogenated anesthetic gases determined at each time interval and on the basis of the predetermined retention capacity of the filter 302, 402.

Advantageously, the calculating means are configured to calculate the remaining organohalogen concentration retention capacity of the filter 302, 402 at a predetermined time, as well as the estimated remaining usage time of the filter 302, 402.

Advantageously, anesthesia station 1 comprises means for displaying calculated data, particularly data relating to the saturation rate of filter 302, 402, the remaining concentration retention capacity, and/or the estimated remaining usage time of the filter 302, 402. To refine the timekeeping of the usage rate, a flow meter may be installed in the aspiration lines, at the output of the filter 302, 402, in order to measure the total gaseous volume filtered by the filter 302, 402.

Advantageously, the anesthesia station 1 comprises visual and/or sound-based alert means coupled to the calculation unit.

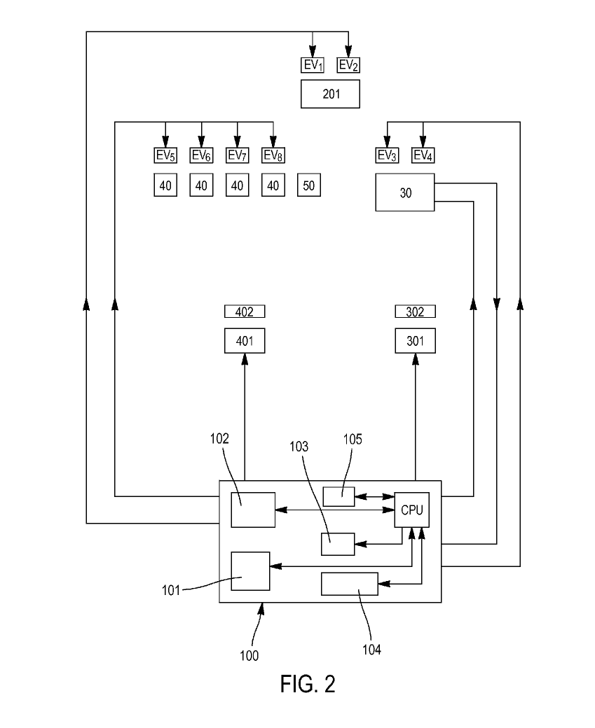

The anesthesia unit 1 also comprises a control unit 100 integrated in the anesthesia area 2. As indicated above and depicted in FIG. 2, the control unit 100 is configured to control the operation of the distribution point 200 (control of the injection of the air or anesthetic gases into the peripherals, control of the flow of air and gas), but also to control the operation of the peripherals and of the aspiration/filtration points 300, 400 (control of the aspiration power to be implemented at the level of the induction chamber 30, the masks 40, and the suction plate 50).

As illustrated in FIG. 2, the control unit 100 comprises an RFID interface 101 designed to communicate with the RFID tags attached to the filters 302 and 402, a module 102 for controlling the pressure and flow of the anesthetic gas, a module 103 for controlling the solenoid valves, a module 104 for acquiring and displaying data on the touchscreen, and a memory block 105 storing all data relating to preceding anesthesia sessions—and particularly to the use of the station (for example, the duration of the phases, the amount of gas administered, and so forth).

Regarding the control of the peripherals 3, 4, and in particular the control of the induction chamber 30, the control unit 100 controls the locking or unlocking of the door of the induction chamber 30 depending on whether or not the associated filter 302 is detected by the RFID interface 301. Indeed, the door (or lid) of the induction chamber 30 is locked during the injection phase. The control unit 100 therefore has the function of ending the induction phase (stopping the injection of the gases from the anesthesia area 2), but also of simultaneously triggering the flushing phase (injection of air alone, and aspiration from the station) before unlocking the door and allowing it to be opened by the user. This sequence serves to ensure that the induction chamber 30 is completely flushed before being opened, and therefore ensures that the user will not be exposed to the anesthetic gases when the induction chamber 30 is opened.

The control unit 100 is also configured so as to control the locking or unlocking of the door of the induction chamber 30 in accordance with the saturation rate of the filter 301.

Regarding the control of the distribution point 200, the control unit 100 controls the stopping of the delivery of the halogenated anesthetic gases when, in particular, the calculated saturation rate of the filter 301 corresponds to the maximum saturation rate thereof.

The operation of the anesthesia station 1 is as follows.

Prior to the start of anesthesia, the user uses the acquisition means provided on the anesthesia area 1 to enter the data relating to the type of animal intended to be anesthetized, and, where appropriate, other information such as the number of animals placed in the induction chamber 30, the weight of said animals, and the like. On the basis of this information, the anesthesia station then determines, via the control unit 100, the optimum volume to inject into the injection chamber, and the period for which the injection should be performed, as well as the optimum volume that should be aspirated once the induction phase is concluded, along with the duration during which the aspiration should be carried out. This operation may be performed before or after the rodents have been placed in the induction chamber 30.

When the command to start the induction phase is received, before distributing the anesthetic gases in accordance with the previously determined optimum volume the control unit 100 will initiate an operation to verify the presence of the filter 302 and proper functioning thereof by sending a signal from the RFID interface to the RFID tag carried by the filter 302. If there is no response from the filter, or if a filter saturated with organohalogens is detected, the control unit 100 will prohibit the distribution point 200 from distributing any anesthetic gas at all. If the presence of the filter 302 is detected and the absence of any sign that the filter 302 is saturated is detected, the control unit 100 sends instructions to the distribution point 200 to distribute the anesthetic gas in accordance with the previously determined injection volume.

When the induction phase has been completed, it is followed by the phase for flushing the induction chamber 30—the anesthetic gases are aspirated by the aspiration point associated with the aspiration chamber while, simultaneously, oxygen is injected from the distribution point 200 via bypass 6 into the induction chamber 30. The control unit 100 maintains the locking of the door (or lid) until the flushing phase is completed. The control unit 100 does not send instructions to unlock the door (or lid) of the induction chamber 30 until the flushing phase is completed. This complete sequence thus ensures that the induction chamber 30 is fully flushed, and thus ensures that the user will not be exposed to the anesthetic gases.

The animals are then fitted with masks 40 for the purpose of the maintenance phase. As for the induction chamber 30, the control unit 100 will perform an operation for verifying the presence of the filter 402 associated with the second aspiration/filtration point 400 (filter associated with the masks 40 and the suction plate 50) and proper functioning thereof by sending a signal from the RFID interface to the RFID tag carried by said filter 402. If there is no response from the filter, or if a filter saturated with organohalogens is detected, the control unit 100 will prevent the distribution point 200 from distributing any anesthetic gas at all to the masks 40. If the presence of the filter 402 is detected and the absence of any sign that the filter 402 is saturated is detected, the control unit 100 sends instructions to the distribution point 200 to distribute the anesthetic gas to the masks 40 in accordance with the previously determined injection volume. This volume is determined in advance by the control unit 100 depending on the type of animal. In addition, the control unit 100 also determines the aspiration power to be implemented simultaneously with the injection of the gases. This power is established in accordance with the injection volume of the anesthetic gases into the masks. This in turn ensures optimum capture of the anesthetic gases at the level of the masks 40, and thus ensures that the users will not be exposed to said gases.

Throughout the duration of the induction and maintenance phases, the organohalogen saturation rate of the filters 302, 402 is indicated to the user. To determine the organohalogen saturation rate of the filter 302, 402, through which the halogenated anesthetic gases flow, the procedure is as follows.

First, the flow of the halogenated anesthetic gases passing through the filter 302, 402 is measured periodically. The data collected is then transmitted to the control unit 100, which will determine the concentration of gas retained in the filter 302, 402 with the aid of a chart that has been set up in advance and stored in a memory of the control unit 100. The chart defines a gas concentration in accordance with the flow collected. Generally, the gas flow rate—and thus that of the fan—varies depending on the peripheral used. Thus, conventionally, the gas flow rate is higher when the gas is sent to the induction chamber 30 than the gas flow rate that is sent to the masks. Of course, the flow rate is modified according to the type and size of the animal to be anesthetized. A concentration rate is associated with each flow rate. Thus, the concentration retained by the filter for an induction phase flow rate is in the order of 4%, and for a maintenance phase flow rate the concentration rate is between 1% and 3% (variable depending on the type and size of the animal). It should also be clear that when the flowmeter does not detect a flow, anesthesia station 1 is stopped.

The saturation rate of the filter 302, 402 is determined by summing the gas concentration rate as determined at each time interval, and comparing this with the overall retention capacity of the filter 302, 402. Preferably, the concentration rate is incremented for each new measurement of the gas flow rate. The saturation rate of the filter 302, 402 is displayed on the display screen of the anesthesia area 2. In addition thereto or alternatively thereto, it may be provided to display the remaining organohalogen concentration retention capacity as established on the basis of the organohalogen concentration retention capacity and the saturation rate of the filter 302, 402 calculated at time t.

The control unit 100 then estimates the remaining usage time of the filter 302, 402. More particularly, with the knowledge of the remaining organohalogen concentration retention capacity and the mean flow rate of the halogenated anesthetic gases, which is calculated on the basis of the instantaneous flow rates of the halogenated gases measured at each time interval, the usage time is estimated on the basis of the data stored in memory block 105 and relates to the previous uses of the station (an assessment of the recurrence of use). Thus, for a given remaining organohalogen concentration retention capacity of the filter, the remaining usage time may be deemed insufficient or not in accordance with the previous uses of the station.

The aspiration power to be implemented at the level of the masks 40 is determined by the control unit 100 on the basis of the volume of anesthetic gas that is injected. This ensures optimum capture of the anesthetic gases at the level of masks 40, and ensures that the users will not be exposed to the gases.

The preceding description of the invention is purely exemplary in nature. It will be readily understood that a person skilled in the art would be capable of implementing different embodiments of the invention without departing from the scope of the invention.

1. An anesthesia station for one or more laboratory animals, comprising:
an anesthesia area connected to at least one peripheral receiving the laboratory animal, said anesthesia area being able to deliver halogenated anesthetic gases to said peripheral;
a system for aspirating halogenated gases from the peripheral;
at least one filter having a predetermined organohalogen concentration retention capacity, said filter being arranged in such a way as to be crossed by the halogenated anesthetic gases originating from the peripheral during operation of the aspirating system; and
a system for automatically determining, in real time, the organohalogen saturation rate of the filter, said system comprising:
a device for measuring instantaneous flow rates of the halogenated anesthetic gases passing through the filter, at given time intervals;
a device for determining the concentration of the halogenated gases retained in the filter, on the basis of the instantaneous flow rates of the halogenated anesthetic gases measured at each given time interval; and,
a control unit for calculating the saturation rate of the filter on the basis of the concentrations of the halogenated anesthetic gases determined at each time interval and on the basis of the predetermined retention capacity of the filter.

2. The anesthesia station according to claim 1, wherein the calculating device is configured to calculate the remaining organohalogen concentration capacity of the filter at a given moment.

3. The anesthesia station according to claim 1, wherein the calculating device is configured to estimate a remaining usage time of the filter on the basis of a history of usage data that is recorded in a memory of the control unit of the anesthesia station.

4. The anesthesia station according to claim 1, wherein the station further comprises display screen relating to the saturation rate of the filter, and the remaining organohalogen concentration retention capacity of the filter and/or the estimated remaining usage time of the filter.

5. The anesthesia station according to claim 1, wherein the anesthesia area is connected to at least one peripheral device for distributing halogenated anesthetic gases.

6. The anesthesia station according to claim 1, wherein the aspirating system and the filter are integrated in the anesthesia area.

7. The anesthesia station according to claim 1, wherein the station further comprises a first type of peripheral, called an “induction” peripheral, which has the form of a hermetically sealed chamber.

8. The anesthesia station according to claim 7, wherein the station further comprises a second type of peripheral, called a “maintenance” peripheral, which has the form of a mask designed to receive a previously anesthetized laboratory animal.

9. The anesthesia station according to claim 7, wherein the station further comprises at least one additional peripheral connected to a suction plate for locally aspirating the halogenated gases at a level of a peripheral of the first type or of the second type, or an optical imager.

10. The anesthesia station according to claim 1, wherein the control unit controls the operation of the anesthesia area, said control unit issuing a command to stop delivery of the halogenated anesthetic gases when the calculated saturation rate of the filter corresponds to the maximum saturation rate of the filter.

11. The anesthesia station according to claim 10, wherein the control unit comprises a device for calculating the aspiration power delivered by the aspirating system, in accordance with the volume of anesthetic gas delivered to the peripheral.

12. The anesthesia station according to claim 10, wherein the control unit comprises an RFID reader designed to communicate with an RFID tag carried by the filter.

13. The anesthesia station according to claim 11, wherein the anesthesia area comprises a visual and/or sound-based alert coupled to the calculation device.

14. A method for determining a organohalogen saturation rate of a filter through which halogenated anesthetic gases flow, the filter having a predetermined organohalogen concentration retention capacity, wherein it comprises the steps of:
measuring the flow of halogenated anesthetic gases at regular time intervals;
determining the concentration of gas retained in the filter, by correlation to the flow measured at each time interval; and
calculating the saturation rate of the filter by summing the gas concentrations determined at each interval of time and comparing these with the predetermined retention capacity of the filter.

15. The method for determining the halogen saturation rate of a filter according to claim 14, wherein the method further comprises a step for determining the remaining organohalogen concentration retention capacity on the basis of data relating to the saturation rate of the filter calculated at a time, and to the organohalogen concentration retention capacity.

16. The method for determining the halogen saturation rate of a filter according to claim 15, wherein the method comprises a step for determining the remaining usage time estimated on the basis of data relating to the remaining organohalogen concentration retention capacity and to the mean flow rate of the halogenated anesthetic gases calculated on the basis of the instantaneous flow rates of the halogenated gases measured at each time interval.

17. The method for determining the halogen saturation rate of a filter according to claim 14, wherein the saturation rate of the filter is calculated on the basis of the sum of the concentration of gas determined at a given instant when the instantaneous flow is measured, with incremented concentrations of the gases determined at each time interval.
